SEQUENCE LISTING (20 sequences)

SEQ ID NO: 1 - protein, 325 aa, Lactobacillus plantarum
MDLFESLAQKITGKDQTIVFPEGTEPRIVGAAARLAADGLVKPIVLGATDKVQAVANDLNADLTGVQVLDPATYPAEDKQAMLDALVERRKGKNTPEQAAKMLEDENYFGTMLVYMGKADGMVSGAIHPTGDTVRPALQIIKTKPGSHRISGAFIMQKGEERYVFADCAINIDPDADTLAEIATQSAATAKVFDIDPKVAMLSFSTKGSAKGEMVTKVQEATAKAQAAEPELAIDGELQFDAAFVEKVGLQKAPGSKVAGHANVFVFPELQSGNIGYKIAQRFGHFEAVGPVLQGLNKPVSDLSRGCSEEDVYKVAIITAAQGLA

SEQ ID NO: 2 - protein, 325 aa, Lactobacillus pentosus
MDLFESLSQKITGQDQTIVFPEGTEPRIVGAAARLAADGLVKPIVLGATDKVQAVAKDLNADLAGVQVLDPATYPAEDKQAMLDSLVERRKGKNTPEQAAKMLEDENYFGTMLVYMGKADGMVSGAVHPTGDTVRPALQIIKTKPGSHRISGAFIMQKGEERYVFADCAINIDPDADTLAEIATQSAATAKVFDIDPKVAMLSFSTKGSAKGDMVTKVQEATAKAQAAAPELAIDGEMQFDAAFVEKVGLQKAPGSKVAGHANVFVFPELQSGNIGYKIAQRFGHFEAVGPVLQGLNKPVSDLSRGCSEEDVYKVAIITAAQGLA

SEQ ID NO: 3 - protein, 325 aa, Lactobacillus sp.
MELFDSLKQKINGQNKTIVFPEGADKRVLGAASRLAHDGLIKAIVLGKQAEIDATAKENNIDLSQLTLLDPENIPADQHKAMLDALVERRHGKNTPEQAAEMLKDPNYIGTMMVYMDQADGMVSGAIHATGDTVRPALQIIKTKEGVRRISGAFIMQKGDQRYVFADCAINIELDAAGMAEVAVESAHTAKVFDIDPKVALLSFSTKGSAKGDMVTKVQEATKIAHETAPDLAVDGELQFDAAFVPTVAAQKAPGSDVAGHANVFVFPELQSGNIGYKIAQRFGGFEAIGPILQGLNKPVSDLSRGCNEEDVYKVAIITAAQALN

SEQ ID NO: 4 - protein, 324 aa, Lactobacillus brevis
MELFDSLKQKINGQNKTIVFPEGEDERVLGAASRLVADGLVKAIVLGKQSQIETTATNHAIDLSQLTILDPAQMPSDQHQAMLDALVERRKGKNTPEQAAEMLKDPNYVGTMMVYMGQADGMVSGAVHATGDTVRPALQIIKTKAGVHRISGAFIMQKGDERYVFADCAINIELDAAGMAEVAIESAHTAKVFDIDPKVAMLSFSTKGSAKGDMVTKVQEATALAHESAPDLPLDGELQFDAAFVPNVGTQKAPDSKVAGHANVFVFPELQSGNIGYKIAQRFGGFEAIGPILQGLNKPVSDLSRGCNEEDVYKVAIITAAQSL

SEQ ID NO: 5 - protein, 326 aa, Lactobacillus sp.
MDLFASLAKKITGQNKTIVFPEGTEPRIVGAAARLAADGLVKPIILGDQAKVEAVAKDLNADLTGVQVLDPATYPAAEKQAMLDAFVERRKGKNTPEQAAEMLADANYFGTMLVYLGQADGMVSGAVHSTGDTVRPALQIIKTKPGSHRISGAFIMQKGDERYVFADCAINIDPDADTLAEIATQSAHTAKIFDIDPRVAMLSFSTKGSAKGDMVTKMQEATAKAQAADPELAIDGELQFDAAFVEKVGLQKAPGSKVAGHANVFVFPELQSGNIGYKIAQRFGGFEAVGPILQGLNKPVSDLSRGASEEDVYKVAIITAAQGLDA

SEQ ID NO: 6 - protein, 324 aa, Lactobacillus fermentum
MDIFEKLADQLRGQDKTIVFPEGEDPRVLGAAIRLKKDQLVEPVVLGNQEAVEKVAGENGFDLTGLQILDPATYPAEDKQAMHDALLERRNGKNTPEQVDQMLEDISYFATMLVYMGKVDGMVSGAVHATGDTVRPALQIIKTKPGSHRISGAFIMQKGEERYVFADCAINIELDASTMAEVASQSAETAKLFGIDPKVAMLSFSTKGSAKGDMVTKVAEATKLAKEANPDLAIDGELQFDAAFVPSVGELKAPGSDVAGHANVFIFPSLEAGNIGYKIAQRFGGFEAIGPVLQGLNAPVADLSRGTDEEAVYKVALITAAQAL

SEQ ID NO: 7 - protein, 325 aa, Lactobacillus sp.
MDLFESLAKKITGKDQTIVFPEGTEPRIVGAAARLAADGLVQPIVLGAADKIQAVAKELNADLTGVQVLDSATYPDADKKAMLDALVDRRKGKNTPEQATKMLEDPNYFGTMLVYMGKADGMVSGAVHPTGDTVRPALQIIKTKPGSHRISGAFIMQKGEERYVFADCAINIDPDADTLAEIATQSAATAKVFDIEPKVAMLSFSTKGSAKGDMVTKVQEATAKAQAAAPELAIDGELQFDAAFVEKVGLQKAPGSKVAGHANVFVFPELQSGNIGYKIAQRFGHFEAVGPVLQGLNKPVSDLSRGCSEEDVYKVAIITAAQGLA

SEQ ID NO: 8 - protein, 324 aa, Lactobacillus sp.
MDLFEGLASKIKGQDKTLVFPEGEDKRIQGAAIRLKADGLVQPVLLGDQAQIEQTANENNFDLSGIQVIDPANFPEDKKQAMLDALVDRRKGKNTPEQAAEMLKDVSYFGTMLVYMNEVDGMVSGAVHPTGDTVRPALQIIKTKPGSKRISGAFVMQKGDTRLVFADCAINIELDAPTMAEVALQSAHTAKMFDIDPKVALLSFSTKGSAKGEMVTKVAEATKLAHEGDPKLALDGELQFDAAFVESVGEQKAPGSAVAGHANVFVFPDLQSGNIGYKIAQRLGGFEAVGPILQGLNAPISDLSRGASEEDVYKVALITAAQSI

SEQ ID NO: 9 - protein, 325 aa, Lactobacillus sp.
MDLFTSLAQKITGKDQTIVFPEGTEPRIVGAAARLAADGLVKPIVLGATDKVQAVAKDLKADLSGVQVLDPATYPAADKQAMLDSLVERRKGKNTPEQAAKMLEDENYFGTMLVYMGKADGMVSGAVHPTGDTVRPALQIIKTKPGSHRISGAFIMQKGDERYVFADCAINIDPDADTLAEIATQSAHTAEIFDIDPKVAMLSFSTKGSAKGDMVTKVQEATAKAQAAEPDLAIDGELQFDAAFVEKVGLQKAPGSKVAGHANVFVFPELQSGNIGYKIAQRFGGFEAVGPILQGLNKPVSDLSRGASEEDVYKVAIITAAQGLA

SEQ ID NO: 10 - DNA, 978 bp, Artificial Sequence
  codon-optimized DNA sequence
atggacttgttcgaatctttggctcaaaagatcactggtaaggaccaaactatcgtcttcccagaaggtaccgaaccaagaattgttggtgctgccgctagattggctgccgatggtttggtcaagccaatcgttttgggtgctaccgacaaggtccaagctgttgccaacgacttgaacgccgacttgactggtgttcaagtcttagatccagctacctaccctgccgaagacaagcaagctatgttggatgctttggtcgaaagacgtaagggtaagaacactccagaacaagctgccaagatgttggaagacgaaaactactttggtaccatgttggtttacatgggcaaggccgatggtatggtctctggtgctattcacccaactggtgataccgtcagaccagctttacaaattatcaagaccaaaccaggttctcacagaatctccggtgctttcattatgcaaaagggtgaagagagatacgtttttgctgactgtgccatcaacatcgacccagatgctgacaccctagctgaaattgctactcaatctgctgccactgccaaggtcttcgacattgatccaaaggttgctatgttgtctttttccaccaagggttctgccaagggtgaaatggtcaccaaggttcaagaagctactgctaaggctcaagctgccgaaccagaattggctatcgacggtgaattacaattcgacgctgccttcgtcgaaaaggttggtttgcaaaaggctcctggttccaaggttgctggtcacgctaacgtcttcgtttttccagaattgcaatctggcaatatcggttacaagattgctcaaagatttggtcacttcgaagctgtcggtccagttctacaaggtttgaacaaaccagtctccgacttgtctagaggttgttctgaagaggacgtctacaaggttgctatcattactgctgcccaaggtttggcttaa

SEQ ID NO: 11 - DNA, 978 bp, Artificial Sequence
  codon-optimized DNA sequence based on L. pentosus
atggacttgttcgaatctttgtcccaaaagatcactggtcaagaccaaactatcgtcttcccagaaggtaccgaaccaagaattgttggtgctgccgctagattggctgccgatggtttggtcaagccaatcgttttgggtgctaccgacaaggtccaagctgttgccaaggacttgaacgccgacttggctggtgttcaagtcttagatccagctacctaccctgccgaagacaagcaagctatgttggattctttggtcgaaagacgtaagggtaagaacactccagaacaagctgccaagatgttggaagacgaaaactactttggtaccatgttggtttacatgggcaaggccgatggtatggtctctggtgctgttcacccaactggtgataccgtcagaccagctttacaaattatcaagaccaaaccaggttctcacagaatctccggtgctttcattatgcaaaagggtgaagagagatacgtttttgctgactgtgccatcaacatcgatccagatgctgacaccctagctgaaattgctactcaatctgctgccactgccaaggtcttcgacattgatccaaaggttgctatgttgtctttttccaccaagggttctgccaagggtgatatggtcaccaaggttcaagaagctactgctaaggctcaagctgccgctccagaattggctatcgacggtgaaatgcaattcgacgctgccttcgtcgaaaaggttggtttgcaaaaggctcctggttccaaggttgctggtcacgctaacgtcttcgtttttccagaattgcaatctggcaatatcggttacaagattgctcaaagatttggtcacttcgaagctgtcggtccagttctacaaggtttgaacaaaccagtctccgacttgtctagaggttgttctgaagaggacgtctacaaggttgctatcattactgctgcccaaggtttggcttaa

SEQ ID NO: 12 - DNA, 978 bp, Artificial Sequence
  codon-optimized DNA sequence based on L. acidifarinae
atggaattgttcgactctttgaagcaaaagatcaacggtcaaaacaagactatcgtcttcccagaaggtgctgacaagagagttttgggtgctgcctccagattggctcacgatggtttgatcaaggctatcgttttgggtaagcaagctgaaatcgacgctactgccaaggaaaacaacatcgacttgtctcaattgaccctattagatccagaaaacattcctgccgaccaacacaaggctatgttggatgctttggtcgaaagacgtcacggtaagaacactccagaacaagctgccgaaatgttgaaggacccaaactacatcggtaccatgatggtttacatggaccaagccgatggtatggtctctggtgctattcacgctactggtgataccgtcagaccagctttacaaattatcaagaccaaagaaggtgtcaggagaatctccggtgctttcattatgcaaaagggtgaccaaagatacgtttttgctgactgtgccatcaacatcgaattggatgctgctggtatggctgaagttgctgtcgaatctgctcacactgccaaggtcttcgacattgatccaaaggttgctttattgtctttttccaccaagggttctgccaagggtgacatggtcaccaaggttcaagaagctactaagattgctcacgaaactgctccagacttggctgttgacggtgaattacaattcgacgctgccttcgtcccaaccgttgctgcccaaaaggctcctggttccgacgttgctggtcacgctaacgtcttcgtttttccagaattgcaatctggcaatatcggttacaagattgctcaaagatttggtggtttcgaagctatcggtccaattctacaaggtttgaacaaaccagtctccgacttgtctagaggttgtaacgaagaggacgtctacaaggttgctatcattactgctgcccaagccttgaactaa

SEQ ID NO: 13 - DNA, 975 bp, Artificial Sequence
  codon-optimized DNA sequence based on L. brevis
atggaattgttcgactctttgaagcaaaagatcaacggtcaaaacaagactatcgtcttcccagaaggtgaagacgaaagaatcttgggtgctgcctccagattggttgccgatggtttggtcaaggctatcgttttgggtaagcaatctcaaatcgaaaccactgccaccaaccacgctatcgacttgtctcaattgactatcttagatccagctcaaatgccttccgatcaacaccaagctatgttggatgctttggtcgaaagacgtaagggtaagaacactccagaacaagctgccgaaatgttgaaggacccaaactacgtcggtaccatgatggtttacatgggccaagccgatggtatggtctctggtgctgttcacgctactggtgataccgtcagaccagctttacaaattatcaagaccaaagctggtgttcacagaatctccggtgctttcattatgcaaaagggtgacgagagatacgtttttgctgactgtgccatcaacatcgaattggatgctgctggtatggctgaagttgctatcgaatctgctcacactgccaaggtcttcgacattgatccaaaggttgctatgttgtctttttccaccaagggttctgccaagggtgacatggtcaccaaggttcaagaagctactgctttggctcacgaatctgctccagacttgccattggacggtgaattacaattcgacgctgccttcgtcccaaacgttggtactcaaaaggctcctgactccaaggttgctggtcacgctaacgtcttcgtttttccagaattgcaatctggcaatatcggttacaagattgctcaaagatttggtggcttcgaagctattggtccaatcctacaaggtttgaacaaaccagtctccgacttgtctagaggttgtaacgaagaggacgtctacaaggttgctatcattactgctgcccaatccttgtaa

SEQ ID NO: 14 - DNA, 975 bp, Artificial Sequence
  codon-optimized DNA sequence based on L. fabifermentans
atggacatcttcgaaaagttggctgaccaattgagaggtcaagacaagactatcgtcttcccagaaggtgaagacccaagagttttgggtgctgccatcagattgaaaaaggatcaattggtcgaaccagtcgttttgggtaaccaagaagctgtcgaaaaggttgccggtgaaaacggtttcgacttgactggtttgcaaatcttagatccagctacctaccctgccgaagacaagcaagctatgcacgatgctttattggaaagacgtaacggtaagaacactccagaacaagtcgatcaaatgttggaagacatctcttactttgctaccatgttggtttacatgggcaaggtcgatggtatggtttctggtgctgtccacgctactggtgataccgtcagaccagctttacaaattatcaagaccaaaccaggttctcacagaatctccggtgctttcattatgcaaaagggtgaagagagatacgtttttgctgactgtgccatcaacatcgaattggatgcttctaccatggctgaagttgcttcccaatctgctgaaactgccaagttgttcggtattgatccaaaggttgctatgttgtctttttccaccaagggttctgccaagggtgacatggtcaccaaggttgctgaagctaccaagttggctaaggaagccaacccagacttggctatcgacggtgaattacaattcgacgctgccttcgtcccatctgttggcgaattgaaggctcctggttccgacgttgctggtcacgctaacgtcttcatctttccatctttggaagctggcaatatcggttacaagattgctcaaagatttggtggcttcgaagctatcggtccagttctacaaggtttgaacgctccagtcgccgacttgtctagaggtactgacgaagaggctgtctacaaggttgctttgattactgctgcccaagctctataa

SEQ ID NO: 15 - DNA, 978 bp, Artificial Sequence
  codon-optimized DNA sequence based on L. fermentum
atggacttgttcgcttctttggctaagaagatcactggtcaaaacaagactatcgtcttcccagaaggtaccgaaccaagaattgttggtgctgccgctagattggctgccgatggtttggtcaagccaatcattttgggtgaccaagccaaggtcgaagctgttgccaaggacttgaacgccgacttgactggtgttcaagtcttagatccagctacctaccctgctgccgaaaagcaagctatgttggatgcttttgtcgaaagacgtaagggtaagaacactccagaacaagctgccgaaatgttggctgacgccaactactttggtaccatgttggtttacttgggccaagccgatggtatggtctctggtgctgttcactccactggtgataccgtcagaccagctttacaaattatcaagaccaaaccaggttctcacagaatctccggtgctttcattatgcaaaagggtgacgagagatacgtttttgctgactgtgccatcaacatcgacccagatgctgacaccctagctgaaattgctactcaatctgctcacactgccaagatcttcgacattgatccaagagttgctatgttgtctttttccaccaagggttctgccaagggtgacatggtcaccaagatgcaagaagctactgctaaggctcaagctgccgatccagaattggctatcgacggtgaattacaattcgacgctgccttcgtcgaaaaggttggtttgcaaaaggctcctggttccaaggttgctggtcacgctaacgtcttcgtttttccagaattgcaatctggcaatatcggttacaagattgctcaaagatttggtggcttcgaagctgtcggtccaattctacaaggtttgaacaaaccagtctccgacttgtctagaggtgcttctgaagaggacgtctacaaggttgctatcattactgctgcccaaggtttggcttaa

SEQ ID NO: 16 - DNA, 978 bp, Artificial Sequence
  codon-optimized DNA sequence based on L. herbarum
atggacttgttcgaatctttggctaagaagatcactggtaaggaccaaactatcgtcttcccagaaggtaccgaaccaagaattgttggtgctgccgctagattggctgccgatggtttggtccaaccaatcgttttgggtgctgccgacaagattcaagctgttgccaaggaattgaacgccgacttgactggtgttcaagtcttagattctgctacctaccctgatgctgacaagaaagctatgttggatgctttggttgacagacgtaagggtaagaacactccagaacaagctaccaagatgttggaagacccaaactactttggtaccatgttggtttacatgggcaaggccgatggtatggtctctggtgctgttcacccaactggtgataccgtcagaccagctttacaaattatcaagaccaaaccaggttctcacagaatctccggtgctttcattatgcaaaagggtgaagagagatacgtttttgctgactgtgccatcaacatcgacccagatgctgacaccctagctgaaattgctactcaatctgctgccactgccaaggtcttcgacattgaaccaaaggttgctatgttgtctttttccaccaagggttctgccaagggtgacatggtcaccaaggttcaagaagctactgctaaggctcaagctgccgctccagaattggctatcgacggtgaattacaattcgacgctgccttcgtcgaaaaggttggtttgcaaaaggctcctggttccaaggttgctggtcacgctaacgtcttcgtttttccagaattgcaatctggcaatatcggttacaagattgctcaaagatttggtcacttcgaagctgtcggtccagttctacaaggtttgaacaaaccagtctccgacttgtctagaggttgttctgaagaggacgtctacaaggttgctatcattactgctgcccaaggtttggcttaa

SEQ ID NO: 17 - DNA, 975 bp, Artificial Sequence
  codon-optimized DNA sequence based on L. suebicus
atggacttgttcgaaggtttggcttccaagatcaagggtcaagacaagactttggtcttcccagaaggtgaagacaagagaatccaaggtgctgccatcagattgaaggccgatggtttggtccaaccagttttattgggtgaccaagctcaaatcgaacaaactgccaacgaaaacaactttgacttgtctggtattcaagtcattgatccagctaactttcctgaagacaaaaagcaagctatgttggatgctttggttgacagacgtaagggtaagaacactccagaacaagctgccgaaatgttgaaggacgtttcttactttggtaccatgttggtttacatgaacgaagtcgatggtatggtctctggtgctgttcacccaactggtgataccgtcagaccagctttacaaattatcaagaccaaaccaggttctaagagaatctccggtgctttcgttatgcaaaagggtgacaccagattggtttttgctgactgtgccatcaacatcgaattggatgctcaaacaatggctgaagttgctttgcaatctgctcacactgccaagatgttcgacattgatccaaaggttgctttattgtctttttccaccaagggttctgccaagggtgaaatggtcaccaaggttgctgaagctactaagttggctcacgaaggcgatccaaagttggctctagacggtgaattacaattcgacgctgccttcgtcgaatctgttggtgaacaaaaggctcctggttccgctgttgctggtcacgctaacgtcttcgtttttccagacttgcaatctggcaatatcggttacaagattgctcaaagattgggtggtttcgaagctgtcggtccaatcctacaaggtttgaacgctccaatctccgacttgtctagaggtgcttctgaagaggacgtctacaaggttgctttgattactgctgcccaatctatttaa

SEQ ID NO: 18 - DNA, 978 bp, Artificial Sequence
  codon-optimized DNA sequence based on L. xiangfangensis
atggacttgttcacttctttggctcaaaagatcactggtaaggaccaaactatcgtcttcccagaaggtaccgaaccaagaattgttggtgctgccgctagattggctgccgatggtttggtcaagccaatcgttttgggtgctaccgacaaggtccaagctgttgccaaggacttgaaggccgacttgtctggtgttcaagtcttagatccagctacctaccctgccgctgacaagcaagctatgttggattctttggtcgaaagacgtaagggtaagaacactccagaacaagctgccaagatgttggaagacgaaaactactttggtaccatgttggtttacatgggcaaggccgatggtatggtctctggtgctgttcacccaactggtgataccgtcagaccagctttacaaattatcaagaccaaaccaggttctcacagaatctccggtgctttcattatgcaaaagggtgacgagagatacgtttttgctgactgtgccatcaacatcgacccagatgctgacaccctagctgaaattgctactcaatctgctcacactgccgaaatcttcgacattgatccaaaggttgctatgttgtctttttccaccaagggttctgccaagggtgacatggtcaccaaggttcaagaagctactgctaaggctcaagctgccgaaccagatttggctatcgacggtgaattacaattcgacgctgccttcgtcgaaaaggttggtttgcaaaaggctcctggttccaaggttgctggtcacgctaacgtcttcgtttttccagaattgcaatctggcaatatcggttacaagattgctcaaagatttggtggcttcgaagctgtcggtccaattctacaaggtttgaacaaaccagtctccgacttgtctagaggtgcttctgaagaggacgtctacaaggttgctatcattactgctgcccaaggtttggcttaa

SEQ ID NO: 19 - DNA, 788 bp, Artificial Sequence
  synthetic DNA
tcatctcgcctcaatcgaaatttatactctagtatctgcgatatcgaacagtccctttatatttacgagacaggttttgtccttcctcccccaccaaaaagacgctataaaatactaaatatatctaatatcgctactgctcaattcacctaacgaatgattaccaccaagcatcaacaccatgtgcataccataccgctaactaaactcaccaacgctggaagcctgaataccaagtatcgaactgaggcccctgtgttaccaatccgtaaaaagtgatggaacccgccgctcgcttccaagagttatcatcatattcttcatcatattcttccatacttaaggtgggtagcgaggacccctcaattcccccacctctctgccagggcgtcatctttttctacaaaagccaggctgagtcacgtcagttgctgaccctgggggctgcattgtttcctacgaattactcatttgtttcgtgcgctttcctattgcgcgcatgactaggatggaaaaaaaaagaagaaaaagaaaagcgttgagtatataataagaaagaagaaaaagtccgagagaaaagaagcacaaaggtttttcgtcgaggaaaacagtaaagtttgatacgcacatcgttgacatcgctgactgcaataggaaactgaaatagacggcaaaccattagttcattcgaaagaacgtattgtcgagaattatcactcactatatcagaaaattgacacacgaattatataaacgaagttatacagaaaaagattaaagaaaagaaa

SEQ ID NO: 20 - DNA, 303 bp, Artificial Sequence
  synthetic DNA
gttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttg

(19) United States
(12) Patent Application Publication
(10) Pub. No.: US 2021/0388397 A1
(43) Pub. Date: Dec. 16, 2021

(54) SELECTED PHOSPHOTRANSACETYLASE GENES FOR INCREASED ETHANOL PRODUCTION IN ENGINEERED YEAST

(71) Applicant: DANISCO US INC, Palo Alto, CA (US)

(72) Inventors: Paula Johanna TEUNISSEN, Palo Alto, CA (US); Geetha VEERAMUTHU, Palo Alto, CA (US); Yehong Jamie WANG, Wilmington, DE (US); Quinn Qun ZHU, West Chester, PA (US)

(21) Appl. No.: 17/279,509

(22) PCT Filed: Sep. 25, 2019
(86) PCT No.: PCT/US2019/052838

§ 371 Date: Mar. 24, 2021

Related U.S. Application Data
(60) Provisional application No. 62/738,598, filed on Sep. 28, 2018.

(51) Int. Cl. C12P 7/06; C12N 9/10; C12N 1/16

(52) U.S. Cl. CPC C12P 7/06; C12Y 203/01008; C12N 1/165; C12N 9/1029

(57) ABSTRACT

Described are compositions and methods relating to phosphotransacetylase (PTA) genes that improve ethanol production in yeast harboring an engineered PKL pathway, and yeast expressing these PTA genes. Such yeast is particularly useful for large-scale ethanol production from starch substrates, where acetate in an undesirable by-product.

### CROSS REFERENCE

The present application claims the benefit of U.S. Provisional Application Ser. No. 62/738,598, filed Sep. 28, 2018, which is hereby incorporated by reference in its entirety.

### TECHNICAL FIELD

The present compositions and methods relate to phosphotransacetylase (PTA) genes that improve ethanol production in yeast harboring an engineered PKL pathway, and yeast expressing these PTA genes. Such yeast is particularly useful for large-scale ethanol production from starch substrates, where acetate in an undesirable by-product.

### BACKGROUND

First-generation yeast-based ethanol production converts sugars into fuel ethanol. The annual fuel ethanol production by yeast is about 90 billion liters worldwide (Gombert, A. K. and van Maris. A. J. (2015) Curr. Opin. Biotechnol. 33:81-86). It is estimated that about 70% of the cost of ethanol production is the feedstock. Since the production volume is so large, even small yield improvements have massive economic impact across the industry.

Ethanol production in engineered yeast cells with a heterologous phosphoketolase (PKL) pathway is higher than in a parental strain without a PKL pathway (see, e.g., WO2015148272; Miasnikov et al.). The PKL pathway consists of phosphoketolase (PKL) and phosphotransacetylase (PTA) to channel carbon flux away from the glycerol pathway and toward the synthesis of acetyl-coA. Two supporting enzymes, acetaldehyde dehydrogenase (AADH) and acetyl-coA synthase (ACS), can help the PKL pathway be more effective.

There is an ongoing need to improve the PKL pathway to further increase ethanol production yield.

### SUMMARY

The present compositions and methods relate to phosphotransacetylase (PTA) genes that improve ethanol production in yeast harboring an engineered PKL pathway, and yeast expressing these PTA genes. Aspects and embodiments of the compositions and methods are described in the following, independently-numbered, paragraphs.

1. In one aspect, modified yeast cells are provided, comprising an exogenous phophoketolase pathway including a phosphotransacetylase gene derived from Lactobacillus acidifarinae, Lactobacillus fabifermentans and/or Lactobacillus xiangfangensis, and/or encoding a polypeptide having at least at least 75% amino acid sequence identity to SEQ ID NO: 3, at least 77% amino acid sequence identity to SEQ ID NO: 5 and/or at least 96% amino acid sequence identity to SEQ ID NO: 9, wherein the phosphotransacetylase gene does not encode a polypeptide identical to the phosphotransacetylase polypeptide from Lactobacillus plantarum having the amino acid sequence of SEQ ID NO: 1.

2. In some embodiments of the modified cells of paragraph 1, the exogenous phophoketolase pathway includes a gene encoding a phosphoketolase and a gene encoding a phosphotransacetylase.

3. In some embodiments of the modified cells of paragraph 2, the exogenous phophoketolase pathway further includes a gene encoding an acetylating acetyl dehydrogenase.

4. In some embodiments, the modified cells of any of paragraphs 1-3 further comprise an exogenous gene encoding a carbohydrate processing enzyme.

5. In some embodiments, the modified cells of any of paragraphs 1-4 further comprise an alteration in the glycerol pathway and/or the acetyl-CoA pathway.

6. In some embodiments, the modified cells of any of paragraphs 1-5 further comprise an alternative pathway for making ethanol.

7. In some embodiments of the modified cells of any of paragraphs 1-6, the cells are of a Saccharomyces spp.

8. In another aspect, a method for increasing the production of ethanol from yeast cells grown on a carbohydrate substrate is provided, comprising: introducing into parental yeast cells an exogenous phophoketolase pathway comprising a phosphotransacetylase gene derived from Lactobacillus acidifarinae, Lactobacillus fabifermentans and/or Lactobacillus xiangfangensis, and/or encoding a polypeptide having at least at least 75% amino acid sequence identity to SEQ ID NO: 3, at least 77% amino acid sequence identity to SEQ ID NO: 5 and/or at least 96% amino acid sequence identity to SEQ ID NO: 9, wherein the phosphotransacetylase gene does not encode a polypeptide identical to the phosphotransacetylase polypeptide from Lactobacillus plantarum having the amino acid sequence of SEQ ID NO: 1.

9. In some embodiments of the method of paragraph 8, the phosphotransacetylase gene is in addition to a phosphotransacetylase gene encoding a phosphotransacetylase polypeptide from Lactobacillus plantarum having the amino acid sequence of SEQ ID NO: 1.

10. In some embodiments of the method of paragraph 8 or 9 the yeast cells are the modified yeast cells of any one of paragraph 1-7.

These and other aspects and embodiments of present compositions and methods will be apparent from the description, including any accompanying Drawings/FIGURES.

### BRIEF DESCRIPTION OF THE DRAWINGS

FIG. 1 is a map of plasmid pZKIIC-62LpP1 used to express PTA.

### DETAILED DESCRIPTION

### I. Definitions

Prior to describing the present yeast and methods in detail, the following terms are defined for clarity. Terms not defined should be accorded their ordinary meanings as used in the relevant art.

As used herein, the term “alcohol” refers to an organic compound in which a hydroxyl functional group (—OH) is bound to a saturated carbon atom.

As used herein, the terms “yeast cells,” “yeast strains,” or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “engineered yeast cells,” “variant yeast cells,” “modified yeast cells,” or similar phrases, refer to yeast that include genetic modifications and characteristics described herein. Variant/modified yeast do not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins,” or “homologs.” Such proteins can be derived from organisms of different genera and/or species, or different classes of organisms (e.g., bacteria and fungi), or artificially designed. Related proteins also encompass homologs determined by primary sequence analysis, determined by secondary or tertiary structure analysis, or determined by immunological cross-reactivity, or determined by their functions.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype. The term “allele” is generally preferred when an organism contains more than one similar genes, in which case each different similar gene is referred to as a distinct “allele.”

As used herein, the term “expressing a polypeptide” and similar terms refers to the cellular process of producing a polypeptide using the translation machinery (e.g., ribosomes) of the cell.

As used herein, “over-expressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes a promoter, and amino acid coding region and a terminator (i.e., promoter::amino acid coding region::terminator) and other nucleic acid sequence needed to allow the encoded polypeptide to be produced in a cell. Expression cassettes can be exogenous (i.e., introduced into a cell) or endogenous (i.e., extant in a cell).

As used herein, the terms “wild-type” and “native” are used interchangeably and refer to genes, proteins or strains found in nature, or that are not intentionally modified for the advantage of the presently described yeast.

As used herein, the term “protein of interest” refers to a polypeptide that is desired to be expressed in modified yeast. Such a protein can be an enzyme, a substrate-binding protein, a surface-active protein, a structural protein, a selectable marker, a signal transducer, a receptor, a transporter, a transcription factor, a translation factor, a co-factor, or the like, and can be expressed. The protein of interest is encoded by an endogenous gene or a heterologous gene (i.e., gene of interest”) relative to the parental strain. The protein of interest can be expressed intracellularly or as a secreted protein.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using CRISPR, RNAi, antisense, or any other method that abolishes gene expression. A gene can be disrupted by deletion or genetic manipulation of non-adjacent control elements. As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements. Deletion of a gene also refers to the deletion a part of the coding sequence, or a part of promoter immediately or not immediately adjacent to the coding sequence, where there is no functional activity of the interested gene existed in the engineered cell.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a binding activity, a surface-active property, a signal transducer, a receptor, a transporter, a transcription factor, a translation factor, a co-factor, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “aerobic fermentation” refers to growth in the presence of oxygen.

As used herein, “anaerobic fermentation” refers to growth in the absence of oxygen.

As used herein, the expression “end of fermentation” refers to the stage of fermentation when the economic advantage of continuing fermentation to produce a small amount of additional alcohol is exceeded by the cost of continuing fermentation in terms of fixed and variable costs. In a more general sense, “end of fermentation” refers to the point where a fermentation will no longer produce a significant amount of additional alcohol, i.e., no more than about 1% additional alcohol, or no more substrate left for further alcohol production.

As used herein, the expression “carbon flux” refers to the rate of turnover of carbon molecules through a metabolic pathway. Carbon flux is regulated by enzymes involved in metabolic pathways, such as the pathway for glucose metabolism and the pathway for maltose metabolism.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:

| | |
|---|---|
| EC | enzyme commission |
| PKL | phosphoketolase |
| PTA | phosphotransacetylase |
| AADH | acetaldehyde dehydrogenases |
| ADH | alcohol dehydrogenase |
| EtOH | ethanol |
| AA | α-amylase |
| GA | glucoamylase |
| ° C. | degrees Centigrade |
| bp | base pairs |
| DNA | deoxyribonucleic acid |
| ds or DS | dry solids |
| g or gm | gram |
| g/L | grams per liter |
| H₂O | water |
| HPLC | high performance liquid chromatography |
| hr or h | hour |
| kg | kilogram |
| M | molar |
| mg | milligram |
| mL or ml | milliliter |
| min | minute |
| mM | millimolar |
| N | normal |
| nm | nanometer |
| PCR | polymerase chain reaction |
| ppm | parts per million |
| rel. | relative |
| Δ | relating to a deletion |
| μg | microgram |
| μL and μl | microliter |
| μM | micromolar |

### II. Phosphotransacetylase Genes for Improving Ethanol Production in Yeast Harboring an Engineered Phosphoketolase Pathway

Described are compositions and methods relating to phosphotransacetylase (PTA) genes that improve ethanol production in yeast harboring an engineered PKL pathway, and yeast expressing these PTA genes. Along with phosphoketolase (PKL), PTA is an essential enzyme in the phosphoketolase pathway, which has been engineered into yeast to improve ethanol production (see, e.g., WO2015148272; Miasnikov et al.).

Perhaps not surprisingly, Applicants have discovered that yeast cells with an extra copy of a PTA expression cassette demonstrate, in cell harboring an engineered PKL pathway, increased PTA enzyme activity and additional ethanol production, compared to otherwise-identical parental cells. However, surprisingly, Applicants have identified at least three PTA genes that demonstrate even greater PTA enzyme activity and additional ethanol production, compared cells with an extra copy of the best described PTA, derived from Lactobacillus plantarum.

PTA that were studied and resulted in the present compositions and methods are described in Table 1.

TABLE 2
PTA characterized herein

| | | Genbank | SEQ |
|---|---|---|---|
| Organism | Abbreviation | Accession No. | ID NO |
| Lactobacillus plantarum | LpPTA1 | WP_003641060 | 1 |
| Lactobacillus pentosus | LpPTA2 | WP_003637888 | 2 |
| Lactobacillus acidifarinae | LaPTA | WP_057800743 | 3 |
| Lactobacillus brevis | LbPTA | WP_043022134 | 4 |
| Lactobacillus fabifermentans | LfPTA1 | WP_024624028 | 5 |
| Lactobacillus fermentum | LfPTA2 | WP_049184857 | 6 |
| Lactobacillus herbarum | LhPTA | WP_04799903 | 7 |
| Lactobacillus suebicus | LsPTA | WP_010622891 | 8 |
| Lactobacillus xiangfangensis | LxPTA | WP_057706802 | 9 |

The amino acid sequence of the LpPTA1 polypeptide from L. plantarum is shown, below, as SEQ ID NO: 1:

MDLFESLAQKITGKDQTIVFPEGTEPRIVGAAARLAADGLVKPIVLGATD
KVQAVANDLNADLTGVQVLDPATYPAEDKQAMLDALVERRKGKNTPEQAA
KMLEDENYFGTMLVYMGKADGMVSGAIHPTGDTVRPALQIIKTKPGSHRI
SGAFIMQKGEERYVFADCAINIDPDADTLAEIATQSAATAKVFDIDPKVA
MLSFSTKGSAKGEMVTKVQEATAKAQAAEPELAIDGELQFDAAFVEKVGL
QKAPGSKVAGHANVFVFPELQSGNIGYKIAQRFGHFEAVGPVLQGLNKPV
SDLSRGCSEEDVYKVAIITAAQGLA

The amino acid sequence of the LpPTA2 polypeptide from L. pentosus is shown, below, as SEQ ID NO: 2:

MDLFESLSQKITGQDQTIVFPEGTEPRIVGAAARLAADGLVKPIVLGATD
KVQAVAKDLNADLAGVQVLDPATYPAEDKQAMLDSLVERRKGKNTPEQAA
KMLEDENYFGTMLVYMGKADGMVSGAVHPTGDTVRPALQIIKTKPGSHRI
SGAFIMQKGEERYVFADCAINIDPDADTLAEIATQSAATAKVFDIDPKVA
MLSFSTKGSAKGDMVTKVQEATAKAQAAAPELAIDGEMQFDAAFVEKVGL
QKAPGSKVAGHANVFVFPELQSGNIGYKIAQRFGHFEAVGPVLQGLNKPV
SDLSRGCSEEDVYKVAIITAAQGLA

The amino acid sequence of the LaPTA polypeptide from L. acidifarinae is shown, below, as SEQ ID NO: 3:

MELFDSLKQKINGQNKTIVFPEGADKRVLGAASRLAHDGLIKAIVLGKQA
EIDATAKENNIDLSQLTLLDPENIPADQHKAMLDALVERRHGKNTPEQAA
EMLKDPNYIGTMMVYMDQADGMVSGAIHATGDTVRPALQIIKTKEGVRRI
SGAFIMQKGDQRYVFADCAINIELDAAGMAEVAVESAHTAKVFDIDPKVA
LLSFSTKGSAKGDMVTKVQEATKIAHETAPDLAVDGELQFDAAFVPTVAA
QKAPGSDVAGHANVFVFPELQSGNIGYKIAQRFGGFEAIGPILQGLNKPV
SDLSRGCNEEDVYKVAIITAAQALN

The amino acid sequence of the LbPTA polypeptide from L. brevis is shown, below, as SEQ ID NO: 4:

MELFDSLKQKINGQNKTIVFPEGEDERVLGAASRLVADGLVKAIVLGKQS
QIETTATNHAIDLSQLTILDPAQMPSDQHQAMLDALVERRKGKNTPEQAA
EMLKDPNYVGTMMVYMGQADGMVSGAVHATGDTVRPALQIIKTKAGVHRI
SGAFIMQKGDERYVFADCAINIELDAAGMAEVAIESAHTAKVFDIDPKVA
MLSFSTKGSAKGDMVTKVQEATALAHESAPDLPLDGELQFDAAFVPNVGT
QKAPDSKVAGHANVFVFPELQSGNIGYKIAQRFGGFEAIGPILQGLNKPV
SDLSRGCNEEDVYKVAIITAAQSL

The amino acid sequence of the LfPTA1 polypeptide from L. fabifermentans is shown, below, as SEQ ID NO: 5:

MDLFASLAKKITGQNKTIVFPEGTEPRIVGAAARLAADGLVKPIILGDQA
KVEAVAKDLNADLTGVQVLDPATYPAAEKQAMLDAFVERRKGKNTPEQAA
EMLADANYFGTMLVYLGQADGMVSGAVHSTGDTVRPALQIIKTKPGSHRI
SGAFIMQKGDERYVFADCAINIDPDADTLAEIATQSAHTAKIFDIDPRVA
MLSFSTKGSAKGDMVTKMQEATAKAQAADPELAIDGELQFDAAFVEKVGL
QKAPGSKVAGHANVFVFPELQSGNIGYKIAQRFGGFEAVGPILQGLNKPV
SDLSRGASEEDVYKVAIITAAQGLDA

The amino acid sequence of the LfPTA2 polypeptide from L. fermentum is shown, below, as SEQ ID NO: 6:

MDIFEKLADQLRGQDKTIVFPEGEDPRVLGAAIRLKKDQLVEPVVLGNQE
AVEKVAGENGFDLTGLQILDPATYPAEDKQAMHDALLERRNGKNTPEQVD
QMLEDISYFATMLVYMGKVDGMVSGAVHATGDTVRPALQIIKTKPGSHRI
SGAFIMQKGEERYVFADCAINIELDASTMAEVASQSAETAKLFGIDPKVA
MLSFSTKGSAKGDMVTKVAEATKLAKEANPDLAIDGELQFDAAFVPSVGE
LKAPGSDVAGHANVFIFPSLEAGNIGYKIAQRFGGFEAIGPVLQGLNAPV
ADLSRGTDEEAVYKVALITAAQAL

The amino acid sequence of the LhPTA polypeptide from L. herbarum is shown, below, as SEQ ID NO: 7:

MDLFESLAKKITGKDQTIVFPEGTEPRIVGAAARLAADGLVQPIVLGAAD
KIQAVAKELNADLTGVQVLDSATYPDADKKAMLDALVDRRKGKNTPEQAT
KMLEDPNYFGTMLVYMGKADGMVSGAVHPTGDTVRPALQIIKTKPGSHRI
SGAFIMQKGEERYVFADCAINIDPDADTLAEIATQSAATAKVFDIEPKVA
MLSFSTKGSAKGDMVTKVQEATAKAQAAAPELAIDGELQFDAAFVEKVGL
QKAPGSKVAGHANVFVFPELQSGNIGYKIAQRFGHFEAVGPVLQGLNKPV
SDLSRGCSEEDVYKVAIITAAQGLA

The amino acid sequence of the LsPTA polypeptide from L. suebicus is shown, below, as SEQ ID NO: 8:

MDLFEGLASKIKGQDKTLVFPEGEDKRIQGAAIRLKADGLVQPVLLGDQA
QIEQTANENNFDLSGIQVIDPANFPEDKKQAMLDALVDRRKGKNTPEQAA
EMLKDVSYFGTMLVYMNEVDGMVSGAVHPTGDTVRPALQIIKTKPGSKRI
SGAFVMQKGDTRLVFADCAINIELDAPTMAEVALQSAHTAKMFDIDPKVA
LLSFSTKGSAKGEMVTKVAEATKLAHEGDPKLALDGELQFDAAFVESVGE
QKAPGSAVAGHANVFVFPDLQSGNIGYKIAQRLGGFEAVGPILQGLNAPI
SDLSRGASEEDVYKVALITAAQSI

The amino acid sequence of the LxPTA polypeptide from L. xiangfangensis is shown, below, as SEQ ID NO: 9:

MDLFTSLAQKITGKDQTIVFPEGTEPRIVGAAARLAADGLVKPIVLGATD
KVQAVAKDLKADLSGVQVLDPATYPAADKQAMLDSLVERRKGKNTPEQAA
KMLEDENYFGTMLVYMGKADGMVSGAVHPTGDTVRPALQIIKTKPGSHRI
SGAFIMQKGDERYVFADCAINIDPDADTLAEIATQSAHTAEIFDIDPKVA
MLSFSTKGSAKGDMVTKVQEATAKAQAAEPDLAIDGELQFDAAFVEKVGL
QKAPGSKVAGHANVFVFPELQSGNIGYKIAQRFGGFEAVGPILQGLNKPV
SDLSRGASEEDVYKVAIITAAQGLA

In some embodiments, the PTA expressed in yeast harboring an engineered PKL pathway is LaPTA (SEQ ID NO: 3), LbPTA (SEQ ID NO: 4), LfPTA1 (SEQ ID NO: 5) and/or LxPTA (SEQ ID NO: 9).

In some embodiments, the PTA expressed in yeast harboring an engineered PKL pathway is at least 75%, at least 80%, at least 85%, at least 90%, at least 95%, or greater, in terms of amino acid sequence identity to SEQ ID NO: 3. In some embodiments, the PTA over-expressed in yeast harboring an engineered PKL pathway is at least 77%, at least 80%, at least 85%, at least 90%, at least 95%, or greater, in terms of amino acid sequence identity to SEQ ID NO: 4. In some embodiments, the PTA expressed in yeast harboring an engineered PKL pathway is at least 77%, at least 80%, at least 85%, at least 90%, at least 95%, or greater, in terms of amino acid sequence identity to SEQ ID NO: 5. In some embodiments, the PTA expressed in yeast harboring an engineered PKL pathway is at least 96%, or greater, in terms of amino acid sequence identity to SEQ ID NO: 9.

In some embodiments, the PTA expressed in yeast harboring an engineered PKL pathway is a functionally and/or structurally similar homologous protein with respect to LaPTA (SEQ ID NO: 3), LbPTA (SEQ ID NO: 4), LfPTA1 (SEQ ID NO: 5) and/or LxPTA (SEQ ID NO: 9), but is not identical to LpPTA1 (SEQ ID NO: 1).

Preferably, increased PTA expression and activity is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the present compositions and methods involve introducing into yeast cells a nucleic acid capable of directing the over-expression, or increased expression, of a PTA polypeptide. Particular methods include but are not limited to (i) introducing an exogenous expression cassette for producing the polypeptide into a host cell, (ii) increase copy number of the same or different cassettes for over-expression of PTA, (iii) modifying any aspect of the host cell to increase the transcription and/or translation, (iv) Modifying any aspect of the host cell to increase the half-life of the PTA transcripts and/or polypeptide in the host cell.

In some embodiments, the parental cell that is modified already includes a gene of interest, such as a gene encoding a selectable marker, carbohydrate-processing enzyme, or other polypeptide. In some embodiments, a gene of introduced is subsequently introduced into the modified cells.

### III. Combination of Increased PTA Production with Other Mutations that Affect Alcohol Production

In some embodiments, in addition to expressing increased amounts and activities of PTA polypeptides in combination with an exogenous PKL pathway, the present modified yeast cells include additional modifications that affect ethanol production.

The modified cells may further include mutations that result in attenuation of the native glycerol biosynthesis pathway and/or reuse glycerol pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.). Methods to enhance the reuse glycerol pathway by over expression of glycerol dehydrogenase (GCY1) and dihydroxyacetone kinase (DAK1) to convert glycerol to dihydroxyacetone phosphate (Zhang et al. (2013) J. Ind. Microbiol. Biotechnol. 40:1153-60).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-CoA. This partially reduces the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lacks a heterologous gene(s) encoding an acetylating acetaldehyde dehydrogenase, a pyruvate-formate lyase or both.

In some embodiments, the present modified yeast cells may further over-express a sugar transporter-like (STL1) polypeptide to increase the uptake of glycerol (see, e.g., Ferreira et al. (2005) Mol. Biol. Cell. 16:2068-76; Dušková et al. (2015) Mol. Microbiol. 97:541-59 and WO 2015023989 A1) to increase ethanol production and reduce acetate.

In some embodiments, the present modified yeast cells further include a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding ADH1, ADH2, ALD6, BDH1, FRA2, GPD2 or YMR226C.

### IV. Combination of Increased Expression and Activity of PTA with Other Beneficial Mutations

In some embodiments, in addition to increased expression and activity of PTA of polypeptides, optionally in combination with other genetic modifications that benefit alcohol production, the present modified yeast cells further include any number of additional genes of interest encoding proteins of interest. Additional genes of interest may be introduced before, during, or after genetic manipulations that result in the increased production of PTA polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### V. Use of the Modified Yeast for Increased Alcohol Production

The present compositions and methods include methods for increasing alcohol production and/or reducing glycerol production, in fermentation reactions. Such methods are not limited to a particular fermentation process. The present engineered yeast is expected to be a “drop-in” replacement for convention yeast in any alcohol fermentation facility. While primarily intended for fuel alcohol production, the present yeast can also be used for the production of potable alcohol, including wine and beer.

### VI. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### VII. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present yeast strains and methods will be apparent to the skilled person in view of the present description. The following examples are intended to further illustrate, but not limit, the compositions and methods.

### EXAMPLES

### Example 1

### Materials and Methods

### Liquefact Preparation:

Liquefact (corn mash slurry) was prepared by adding 600 ppm of urea, 0.124 SAPU/g ds acid fungal protease, 0.33 GAU/g ds variant Trichoderma glucoamylase and 1.46 SSCU/g ds Aspergillus kawachii α-amylase, adjusted to a pH of 4.8 with sulfuric acid.

### AnKom Assays:

300 μL of concentrated yeast overnight culture was added to each of a number ANKOM bottles filled with 50 g prepared liquefact (see above) to a final OD of 0.3. The bottles were then incubated at 32° C. with shaking at 150 RPM for 55 hours.

### HPLC Analysis:

Samples of the cultures from serum vials and AnKom assays were collected in Eppendorf tubes by centrifugation for 12 minutes at 14,000 RPM. The supernatants were filtered using 0.2 μM PTFE filters and then used for HPLC (Agilent Technologies 1200 series) analysis with the following conditions: Bio-Rad Aminex HPX-87H columns, running at a temperature of 55° C. with a 0.6 ml/min isocratic flow in 0.01 N H2SO4 and a 2.5 μl injection volume. Calibration standards were used for quantification of the of acetate, ethanol, glycerol, glucose and other molecules. Unless otherwise indicated, all values are reported in g/L.

### Cell Culture for Phosphotransacetylase Enzyme Assays:

Cells were grown in 25 ml of clarified industrial corn liquefact media for 24 hours at 32° C. Cells were harvested by centrifugation at 3,000 rpm for 5 minutes and the pellets were washed twice with sterile water.

### Cell-Free-Lysate Sample Preparation for Phosphotransacetylase Enzyme Assays

Cell pellets were resuspended in the buffer containing 50 mM Tris-HCl (pH 7.5), 2 mM 4-benzenesulfonyl fluoride hydrochloride and 1 mM dithiothreitol. Cells were disrupted with glass beads using bead beater. Lysed crude cell extracts were centrifuged at 13,000 rpm for 10 min at 4° C. Cell free clarified lysate samples were collected for enzyme activity analysis.

### Total Protein Analysis for Phosphotransacetylase Enzyme Assays

Total protein was measured using the Pierce BCA protein assay kit.

### Phosphotransacetylase Activity Measurement

Materials used for PTA enzyme assays were as follows: L-malate dehydrogenase from pig heart, porcine citrate synthase, NAD hydrate, NADH, coenzyme A trilithium salt, acetyl phosphate lithium potassium salt, L-malate disodium salt (C₄H₆O₅), 4-benzenesulfonyl fluoride hydrochloride and dithiothreitol (Sigma); glass beads (Scientific Industries); and Pierce BCA protein assay kit (Thermo Scientific).

PTA activity measurements were performed in microtiter plates in 200-μ1 volume. The reaction components were 100 mM Tris-HCL (pH 7.5), 10 mM MgCl₂, 10 mM D,L-malic acid, 3 mM NAD⁺, 0.2 mM coenzyme A, 18 U/mL malate dehydrogenase, 3.3 U/mL citrate synthase and 10 mM acetyl-phosphate (Castano-Cerezo et al. (2009) Microbial Cell Factories, 8:1-19). Reactions were initiated by adding 10 μl of cell lysate sample. Enzyme activity was measured as the increase in NADH absorbance at 340 nm and reported as units of PTA. One unit of PTA was defined as the amount of enzyme required for generation of 1 μmol of NADH per min per mg total protein.

### Example 2

### Identification of Eight Candidates Encoding for Phosphotransacetylase

Using the coding sequence of LpPTA1 (SEQ ID NO: 1) as a nucleic acid hybridization probe, eight additional PTA polypeptides were identified in Genbank (SEQ ID NOs: 2-9, Table 1, supra). Amino acid sequence analysis indicated that these additional PTA polypeptides share about 75-97% amino acid sequence identity with L. plantarum PTA (LpPTA1; Table 2). With the exception of LfPTA1 (Knorr et al. (2001) J. Basic Microbiol. 41:339-49), there are no functional studies involving any of the eight PTA polypeptides.

TABLE 2
Amino acid sequence comparison of eight PTA polypeptides with LpPTA1
Percent Identity

| | LpPTA1 | LpPTA2 | LaPTA | LbPTA | LfPTA1 | LfPTA2 | LhPTA | LsPTA | LxPTA |
|---|---|---|---|---|---|---|---|---|---|
| LpPTA1 | *** | 97.2 | 74.5 | 76.2 | 75.9 | 98.9 | 94.8 | 75.6 | 95.1 |
| LpPTA2 | 2.8 | *** | 74.8 | 76.9 | 75.6 | 90.2 | 94.5 | 74.7 | 95.1 |
| LaPTA | 31.3 | 30.8 | *** | 88.0 | 71.9 | 75.7 | 74.2 | 75.3 | 75.1 |
| LbPTA | 28.6 | 27.7 | 13.2 | *** | 73.5 | 77.8 | 75.6 | 76.2 | 77.5 |
| LfPTA1 | 29.1 | 29.5 | 35.2 | 32.8 | *** | 74.1 | 74.7 | 75.9 | 75.0 |
| LfPTA2 | 10.9 | 10.6 | 29.4 | 26.4 | 31.8 | *** | 88.9 | 77.2 | 91.7 |
| LhPTA | 5.4 | 5.8 | 31.7 | 29.5 | 30.9 | 12.0 | *** | 75.3 | 92.3 |
| LsPTA | 29.5 | 30.9 | 30.0 | 28.6 | 29.1 | 27.3 | 30.0 | *** | 75.9 |
| LxPTA | 5.1 | 5.1 | 30.3 | 26.8 | 30.4 | 8.8 | 8.1 | 29.1 | *** |

Percent Diversity

### Example 3

### Plasmid Constructs for Expressing Codon Optimized PTA

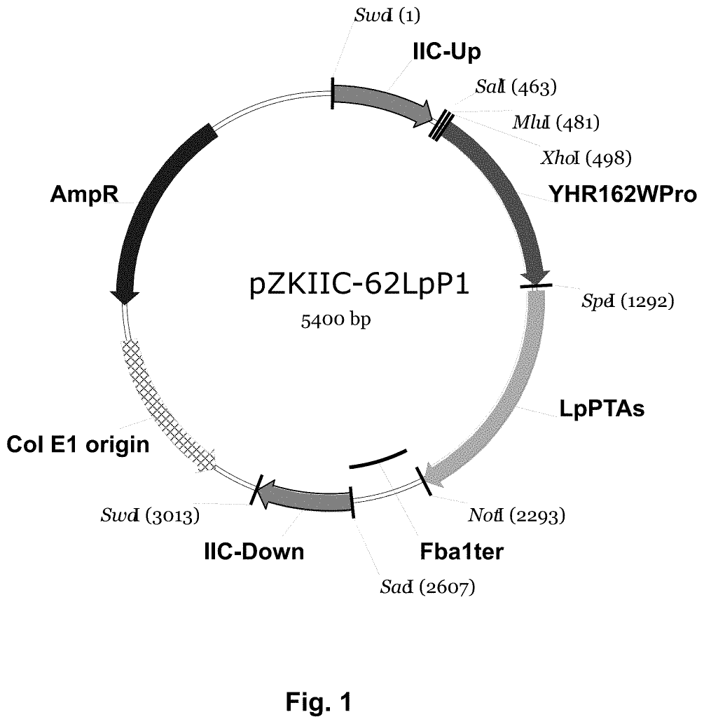

The DNA sequence encoding LpPTA1 (SEQ ID NO: 10) and the other eight PTA molecules (SEQ ID NOs: 11-18) were codon optimized for expression in Saccharomyces cerevisiae and synthesized. Nine constructs were made to separately express the PTA under the control of 62W promoter (S. cerevisiae locus YHR162W, SEQ ID NO: 19) and Fba1 terminator (S. cerevisiae locus YKL060C, SEQ ID NO: 20). All nine constructs were identical to the pZKIIC-62LpP1 plasmid shown in FIG. 1, except that the coding region of LpPTA1s (i.e., SEQ ID NO: 9) was replaced with one of the other eight PTA coding sequences (i.e., SEQ ID NOs: 10-18).

The codon-optimized DNA sequence encoding LpPTA1, derived from L. plantarum, is shown, below, as SEQ ID NO: 10:

ATGGACTTGTTCGAATCTTTGGCTCAAAAGATCACTGGTAAGGACCAAAC
TATCGTCTTCCCAGAAGGTACCGAACCAAGAATTGTTGGTGCTGCCGCTA
GATTGGCTGCCGATGGTTTGGTCAAGCCAATCGTTTTGGGTGCTACCGAC
AAGGTCCAAGCTGTTGCCAACGACTTGAACGCCGACTTGACTGGTGTTCA
AGTCTTAGATCCAGCTACCTACCCTGCCGAAGACAAGCAAGCTATGTTGG
ATGCTTTGGTCGAAAGACGTAAGGGTAAGAACACTCCAGAACAAGCTGCC
AAGATGTTGGAAGACGAAAACTACTTTGGTACCATGTTGGTTTACATGGG
CAAGGCCGATGGTATGGTCTCTGGTGCTATTCACCCAACTGGTGATACCG
TCAGACCAGCTTTACAAATTATCAAGACCAAACCAGGTTCTCACAGAATC
TCCGGTGCTTTCATTATGCAAAAGGGTGAAGAGAGATACGTTTTTGCTGA
CTGTGCCATCAACATCGACCCAGATGCTGACACCCTAGCTGAAATTGCTA
CTCAATCTGCTGCCACTGCCAAGGTCTTCGACATTGATCCAAAGGTTGCT
ATGTTGTCTTTTTCCACCAAGGGTTCTGCCAAGGGTGAAATGGTCACCAA
GGTTCAAGAAGCTACTGCTAAGGCTCAAGCTGCCGAACCAGAATTGGCTA
TCGACGGTGAATTACAATTCGACGCTGCCTTCGTCGAAAAGGTTGGTTTG
CAAAAGGCTCCTGGTTCCAAGGTTGCTGGTCACGCTAACGTCTTCGTTTT
TCCAGAATTGCAATCTGGCAATATCGGTTACAAGATTGCTCAAAGATTTG
GTCACTTCGAAGCTGTCGGTCCAGTTCTACAAGGTTTGAACAAACCAGTC
TCCGACTTGTCTAGAGGTTGTTCTGAAGAGGACGTCTACAAGGTTGCTAT
CATTACTGCTGCCCAAGGTTTGGCTTAA

The codon-optimized DNA sequence encoding LpPTA2, derived from L. pentosus, is shown, below, as SEQ ID NO: 11:

ATGGACTTGTTCGAATCTTTGTCCCAAAAGATCACTGGTCAAGACCAAAC
TATCGTCTTCCCAGAAGGTACCGAACCAAGAATTGTTGGTGCTGCCGCTA
GATTGGCTGCCGATGGTTTGGTCAAGCCAATCGTTTTGGGTGCTACCGAC
AAGGTCCAAGCTGTTGCCAAGGACTTGAACGCCGACTTGGCTGGTGTTCA
AGTCTTAGATCCAGCTACCTACCCTGCCGAAGACAAGCAAGCTATGTTGG
ATTCTTTGGTCGAAAGACGTAAGGGTAAGAACACTCCAGAACAAGCTGCC
AAGATGTTGGAAGACGAAAACTACTTTGGTACCATGTTGGTTTACATGGG
CAAGGCCGATGGTATGGTCTCTGGTGCTGTTCACCCAACTGGTGATACCG
TCAGACCAGCTTTACAAATTATCAAGACCAAACCAGGTTCTCACAGAATC
TCCGGTGCTTTCATTATGCAAAAGGGTGAAGAGAGATACGTTTTTGCTGA
CTGTGCCATCAACATCGATCCAGATGCTGACACCCTAGCTGAAATTGCTA
CTCAATCTGCTGCCACTGCCAAGGTCTTCGACATTGATCCAAAGGTTGCT
ATGTTGTCTTTTTCCACCAAGGGTTCTGCCAAGGGTGATATGGTCACCAA
GGTTCAAGAAGCTACTGCTAAGGCTCAAGCTGCCGCTCCAGAATTGGCTA
TCGACGGTGAAATGCAATTCGACGCTGCCTTCGTCGAAAAGGTTGGTTTG
CAAAAGGCTCCTGGTTCCAAGGTTGCTGGTCACGCTAACGTCTTCGTTTT
TCCAGAATTGCAATCTGGCAATATCGGTTACAAGATTGCTCAAAGATTTG
GTCACTTCGAAGCTGTCGGTCCAGTTCTACAAGGTTTGAACAAACCAGTC
TCCGACTTGTCTAGAGGTTGTTCTGAAGAGGACGTCTACAAGGTTGCTAT
CATTACTGCTGCCCAAGGTTTGGCTTAA

The codon-optimized DNA sequence encoding LaPTA, derived from L. acidifarinae, is shown, below, as SEQ ID NO: 12:

ATGGAATTGTTCGACTCTTTGAAGCAAAAGATCAACGGTCAAAACAAGAC
TATCGTCTTCCCAGAAGGTGCTGACAAGAGAGTTTTGGGTGCTGCCTCCA
GATTGGCTCACGATGGTTTGATCAAGGCTATCGTTTTGGGTAAGCAAGCT
GAAATCGACGCTACTGCCAAGGAAAACAACATCGACTTGTCTCAATTGAC
CCTATTAGATCCAGAAAACATTCCTGCCGACCAACACAAGGCTATGTTGG
ATGCTTTGGTCGAAAGACGTCACGGTAAGAACACTCCAGAACAAGCTGCC
GAAATGTTGAAGGACCCAAACTACATCGGTACCATGATGGTTTACATGGA
CCAAGCCGATGGTATGGTCTCTGGTGCTATTCACGCTACTGGTGATACCG
TCAGACCAGCTTTACAAATTATCAAGACCAAAGAAGGTGTCAGGAGAATC
TCCGGTGCTTTCATTATGCAAAAGGGTGACCAAAGATACGTTTTTGCTGA
CTGTGCCATCAACATCGAATTGGATGCTGCTGGTATGGCTGAAGTTGCTG
TCGAATCTGCTCACACTGCCAAGGTCTTCGACATTGATCCAAAGGTTGCT
TTATTGTCTTTTTCCACCAAGGGTTCTGCCAAGGGTGACATGGTCACCAA
GGTTCAAGAAGCTACTAAGATTGCTCACGAAACTGCTCCAGACTTGGCTG
TTGACGGTGAATTACAATTCGACGCTGCCTTCGTCCCAACCGTTGCTGCC
CAAAAGGCTCCTGGTTCCGACGTTGCTGGTCACGCTAACGTCTTCGTTTT
TCCAGAATTGCAATCTGGCAATATCGGTTACAAGATTGCTCAAAGATTTG
GTGGTTTCGAAGCTATCGGTCCAATTCTACAAGGTTTGAACAAACCAGTC
TCCGACTTGTCTAGAGGTTGTAACGAAGAGGACGTCTACAAGGTTGCTAT
CATTACTGCTGCCCAAGCCTTGAACTAA

The codon-optimized DNA sequence encoding LbPTA, derived from L. brevis, is shown, below, as SEQ ID NO: 13:

ATGGAATTGTTCGACTCTTTGAAGCAAAAGATCAACGGTCAAAACAAGAC
TATCGTCTTCCCAGAAGGTGAAGACGAAAGAATCTTGGGTGCTGCCTCCA
GATTGGTTGCCGATGGTTTGGTCAAGGCTATCGTTTTGGGTAAGCAATCT
CAAATCGAAACCACTGCCACCAACCACGCTATCGACTTGTCTCAATTGAC
TATCTTAGATCCAGCTCAAATGCCTTCCGATCAACACCAAGCTATGTTGG
ATGCTTTGGTCGAAAGACGTAAGGGTAAGAACACTCCAGAACAAGCTGCC
GAAATGTTGAAGGACCCAAACTACGTCGGTACCATGATGGTTTACATGGG
CCAAGCCGATGGTATGGTCTCTGGTGCTGTTCACGCTACTGGTGATACCG
TCAGACCAGCTTTACAAATTATCAAGACCAAAGCTGGTGTTCACAGAATC
TCCGGTGCTTTCATTATGCAAAAGGGTGACGAGAGATACGTTTTTGCTGA
CTGTGCCATCAACATCGAATTGGATGCTGCTGGTATGGCTGAAGTTGCTA
TCGAATCTGCTCACACTGCCAAGGTCTTCGACATTGATCCAAAGGTTGCT
ATGTTGTCTTTTTCCACCAAGGGTTCTGCCAAGGGTGACATGGTCACCAA
GGTTCAAGAAGCTACTGCTTTGGCTCACGAATCTGCTCCAGACTTGCCAT
TGGACGGTGAATTACAATTCGACGCTGCCTTCGTCCCAAACGTTGGTACT
CAAAAGGCTCCTGACTCCAAGGTTGCTGGTCACGCTAACGTCTTCGTTTT
TCCAGAATTGCAATCTGGCAATATCGGTTACAAGATTGCTCAAAGATTTG
GTGGCTTCGAAGCTATTGGTCCAATCCTACAAGGTTTGAACAAACCAGTC
TCCGACTTGTCTAGAGGTTGTAACGAAGAGGACGTCTACAAGGTTGCTAT
CATTACTGCTGCCCAATCCTTGTAA

The codon-optimized DNA sequence encoding LfPTA1, derived from L. fabifermentans, is shown, below, as SEQ ID NO: 14:

ATGGACATCTTCGAAAAGTTGGCTGACCAATTGAGAGGTCAAGACAAGAC
TATCGTCTTCCCAGAAGGTGAAGACCCAAGAGTTTTGGGTGCTGCCATCA
GATTGAAAAAGGATCAATTGGTCGAACCAGTCGTTTTGGGTAACCAAGAA
GCTGTCGAAAAGGTTGCCGGTGAAAACGGTTTCGACTTGACTGGTTTGCA
AATCTTAGATCCAGCTACCTACCCTGCCGAAGACAAGCAAGCTATGCACG
ATGCTTTATTGGAAAGACGTAACGGTAAGAACACTCCAGAACAAGTCGAT
CAAATGTTGGAAGACATCTCTTACTTTGCTACCATGTTGGTTTACATGGG
CAAGGTCGATGGTATGGTTTCTGGTGCTGTCCACGCTACTGGTGATACCG
TCAGACCAGCTTTACAAATTATCAAGACCAAACCAGGTTCTCACAGAATC
TCCGGTGCTTTCATTATGCAAAAGGGTGAAGAGAGATACGTTTTTGCTGA
CTGTGCCATCAACATCGAATTGGATGCTTCTACCATGGCTGAAGTTGCTT
CCCAATCTGCTGAAACTGCCAAGTTGTTCGGTATTGATCCAAAGGTTGCT
ATGTTGTCTTTTTCCACCAAGGGTTCTGCCAAGGGTGACATGGTCACCAA
GGTTGCTGAAGCTACCAAGTTGGCTAAGGAAGCCAACCCAGACTTGGCTA
TCGACGGTGAATTACAATTCGACGCTGCCTTCGTCCCATCTGTTGGCGAA
TTGAAGGCTCCTGGTTCCGACGTTGCTGGTCACGCTAACGTCTTCATCTT
TCCATCTTTGGAAGCTGGCAATATCGGTTACAAGATTGCTCAAAGATTTG
GTGGCTTCGAAGCTATCGGTCCAGTTCTACAAGGTTTGAACGCTCCAGTC
GCCGACTTGTCTAGAGGTACTGACGAAGAGGCTGTCTACAAGGTTGCTTT
GATTACTGCTGCCCAAGCTCTATAA

The codon-optimized DNA sequence encoding LfPTA2, derived from L. fermentum, is shown, below, as SEQ ID NO: 15:

ATGGACTTGTTCGCTTCTTTGGCTAAGAAGATCACTGGTCAAAACAAGAC
TATCGTCTTCCCAGAAGGTACCGAACCAAGAATTGTTGGTGCTGCCGCTA
GATTGGCTGCCGATGGTTTGGTCAAGCCAATCATTTTGGGTGACCAAGCC
AAGGTCGAAGCTGTTGCCAAGGACTTGAACGCCGACTTGACTGGTGTTCA
AGTCTTAGATCCAGCTACCTACCCTGCTGCCGAAAAGCAAGCTATGTTGG
ATGCTTTTGTCGAAAGACGTAAGGGTAAGAACACTCCAGAACAAGCTGCC
GAAATGTTGGCTGACGCCAACTACTTTGGTACCATGTTGGTTTACTTGGG
CCAAGCCGATGGTATGGTCTCTGGTGCTGTTCACTCCACTGGTGATACCG
TCAGACCAGCTTTACAAATTATCAAGACCAAACCAGGTTCTCACAGAATC
TCCGGTGCTTTCATTATGCAAAAGGGTGACGAGAGATACGTTTTTGCTGA
CTGTGCCATCAACATCGACCCAGATGCTGACACCCTAGCTGAAATTGCTA
CTCAATCTGCTCACACTGCCAAGATCTTCGACATTGATCCAAGAGTTGCT
ATGTTGTCTTTTTCCACCAAGGGTTCTGCCAAGGGTGACATGGTCACCAA
GATGCAAGAAGCTACTGCTAAGGCTCAAGCTGCCGATCCAGAATTGGCTA
TCGACGGTGAATTACAATTCGACGCTGCCTTCGTCGAAAAGGTTGGTTTG
CAAAAGGCTCCTGGTTCCAAGGTTGCTGGTCACGCTAACGTCTTCGTTTT
TCCAGAATTGCAATCTGGCAATATCGGTTACAAGATTGCTCAAAGATTTG
GTGGCTTCGAAGCTGTCGGTCCAATTCTACAAGGTTTGAACAAACCAGTC
TCCGACTTGTCTAGAGGTGCTTCTGAAGAGGACGTCTACAAGGTTGCTAT
CATTACTGCTGCCCAAGGTTTGGCTTAA

The codon-optimized DNA sequence encoding LhPTA, derived from L. herbarum, is shown, below, as SEQ ID NO: 16:

ATGGACTTGTTCGAATCTTTGGCTAAGAAGATCACTGGTAAGGACCAAAC
TATCGTCTTCCCAGAAGGTACCGAACCAAGAATTGTTGGTGCTGCCGCTA
GATTGGCTGCCGATGGTTTGGTCCAACCAATCGTTTTGGGTGCTGCCGAC
AAGATTCAAGCTGTTGCCAAGGAATTGAACGCCGACTTGACTGGTGTTCA
AGTCTTAGATTCTGCTACCTACCCTGATGCTGACAAGAAAGCTATGTTGG
ATGCTTTGGTTGACAGACGTAAGGGTAAGAACACTCCAGAACAAGCTACC
AAGATGTTGGAAGACCCAAACTACTTTGGTACCATGTTGGTTTACATGGG
CAAGGCCGATGGTATGGTCTCTGGTGCTGTTCACCCAACTGGTGATACCG
TCAGACCAGCTTTACAAATTATCAAGACCAAACCAGGTTCTCACAGAATC
TCCGGTGCTTTCATTATGCAAAAGGGTGAAGAGAGATACGTTTTTGCTGA
CTGTGCCATCAACATCGACCCAGATGCTGACACCCTAGCTGAAATTGCTA
CTCAATCTGCTGCCACTGCCAAGGTCTTCGACATTGAACCAAAGGTTGCT
ATGTTGTCTTTTTCCACCAAGGGTTCTGCCAAGGGTGACATGGTCACCAA
GGTTCAAGAAGCTACTGCTAAGGCTCAAGCTGCCGCTCCAGAATTGGCTA
TCGACGGTGAATTACAATTCGACGCTGCCTTCGTCGAAAAGGTTGGTTTG
CAAAAGGCTCCTGGTTCCAAGGTTGCTGGTCACGCTAACGTCTTCGTTTT
TCCAGAATTGCAATCTGGCAATATCGGTTACAAGATTGCTCAAAGATTTG
GTCACTTCGAAGCTGTCGGTCCAGTTCTACAAGGTTTGAACAAACCAGTC
TCCGACTTGTCTAGAGGTTGTTCTGAAGAGGACGTCTACAAGGTTGCTAT
CATTACTGCTGCCCAAGGTTTGGCTTAA

The codon-optimized DNA sequence encoding LsPTA, derived from L. suebicus, is shown, below, as SEQ ID NO: 17:

ATGGACTTGTTCGAAGGTTTGGCTTCCAAGATCAAGGGTCAAGACAAGAC
TTTGGTCTTCCCAGAAGGTGAAGACAAGAGAATCCAAGGTGCTGCCATCA
GATTGAAGGCCGATGGTTTGGTCCAACCAGTTTTATTGGGTGACCAAGCT
CAAATCGAACAAACTGCCAACGAAAACAACTTTGACTTGTCTGGTATTCA
AGTCATTGATCCAGCTAACTTTCCTGAAGACAAAAAGCAAGCTATGTTGG
ATGCTTTGGTTGACAGACGTAAGGGTAAGAACACTCCAGAACAAGCTGCC
GAAATGTTGAAGGACGTTTCTTACTTTGGTACCATGTTGGTTTACATGAA
CGAAGTCGATGGTATGGTCTCTGGTGCTGTTCACCCAACTGGTGATACCG
TCAGACCAGCTTTACAAATTATCAAGACCAAACCAGGTTCTAAGAGAATC
TCCGGTGCTTTCGTTATGCAAAAGGGTGACACCAGATTGGTTTTTGCTGA
CTGTGCCATCAACATCGAATTGGATGCTCAAACAATGGCTGAAGTTGCTT
TGCAATCTGCTCACACTGCCAAGATGTTCGACATTGATCCAAAGGTTGCT
TTATTGTCTTTTTCCACCAAGGGTTCTGCCAAGGGTGAAATGGTCACCAA
GGTTGCTGAAGCTACTAAGTTGGCTCACGAAGGCGATCCAAAGTTGGCTC
TAGACGGTGAATTACAATTCGACGCTGCCTTCGTCGAATCTGTTGGTGAA
CAAAAGGCTCCTGGTTCCGCTGTTGCTGGTCACGCTAACGTCTTCGTTTT
TCCAGACTTGCAATCTGGCAATATCGGTTACAAGATTGCTCAAAGATTGG
GTGGTTTCGAAGCTGTCGGTCCAATCCTACAAGGTTTGAACGCTCCAATC
TCCGACTTGTCTAGAGGTGCTTCTGAAGAGGACGTCTACAAGGTTGCTTT
GATTACTGCTGCCCAATCTATTTAA

The codon-optimized DNA sequence encoding LxPTA, derived from L. xiangfangensis, is shown, below, as SEQ ID NO: 18:

ATGGACTTGTTCACTTCTTTGGCTCAAAAGATCACTGGTAAGGACCAAAC
TATCGTCTTCCCAGAAGGTACCGAACCAAGAATTGTTGGTGCTGCCGCTA
GATTGGCTGCCGATGGTTTGGTCAAGCCAATCGTTTTGGGTGCTACCGAC
AAGGTCCAAGCTGTTGCCAAGGACTTGAAGGCCGACTTGTCTGGTGTTCA
AGTCTTAGATCCAGCTACCTACCCTGCCGCTGACAAGCAAGCTATGTTGG
ATTCTTTGGTCGAAAGACGTAAGGGTAAGAACACTCCAGAACAAGCTGCC
AAGATGTTGGAAGACGAAAACTACTTTGGTACCATGTTGGTTTACATGGG
CAAGGCCGATGGTATGGTCTCTGGTGCTGTTCACCCAACTGGTGATACCG
TCAGACCAGCTTTACAAATTATCAAGACCAAACCAGGTTCTCACAGAATC
TCCGGTGCTTTCATTATGCAAAAGGGTGACGAGAGATACGTTTTTGCTGA
CTGTGCCATCAACATCGACCCAGATGCTGACACCCTAGCTGAAATTGCTA
CTCAATCTGCTCACACTGCCGAAATCTTCGACATTGATCCAAAGGTTGCT
ATGTTGTCTTTTTCCACCAAGGGTTCTGCCAAGGGTGACATGGTCACCAA
GGTTCAAGAAGCTACTGCTAAGGCTCAAGCTGCCGAACCAGATTTGGCTA
TCGACGGTGAATTACAATTCGACGCTGCCTTCGTCGAAAAGGTTGGTTTG
CAAAAGGCTCCTGGTTCCAAGGTTGCTGGTCACGCTAACGTCTTCGTTTT
TCCAGAATTGCAATCTGGCAATATCGGTTACAAGATTGCTCAAAGATTTG
GTGGCTTCGAAGCTGTCGGTCCAATTCTACAAGGTTTGAACAAACCAGTC
TCCGACTTGTCTAGAGGTGCTTCTGAAGAGGACGTCTACAAGGTTGCTAT
CATTACTGCTGCCCAAGGTTTGGCTTAA

The DNA sequence of the 62W promoter is shown, below, as SEQ ID NO: 19:

TCATCTCGCCTCAATCGAAATTTATACTCTAGTATCTGCGATATCGAACA
GTCCCTTTATATTTACGAGACAGGTTTTGTCCTTCCTCCCCCACCAAAAA
GACGCTATAAAATACTAAATATATCTAATATCGCTACTGCTCAATTCACC
TAACGAATGATTACCACCAAGCATCAACACCATGTGCATACCATACCGCT
AACTAAACTCACCAACGCTGGAAGCCTGAATACCAAGTATCGAACTGAGG
CCCCTGTGTTACCAATCCGTAAAAAGTGATGGAACCCGCCGCTCGCTTCC
AAGAGTTATCATCATATTCTTCATCATATTCTTCCATACTTAAGGTGGGT
AGCGAGGACCCCTCAATTCCCCCACCTCTCTGCCAGGGCGTCATCTTTTT
CTACAAAAGCCAGGCTGAGTCACGTCAGTTGCTGACCCTGGGGGCTGCAT
TGTTTCCTACGAATTACTCATTTGTTTCGTGCGCTTTCCTATTGCGCGCA
TGACTAGGATGGAAAAAAAAAGAAGAAAAAGAAAAGCGTTGAGTATATAA
TAAGAAAGAAGAAAAAGTCCGAGAGAAAAGAAGCACAAAGGTTTTTCGTC
GAGGAAAACAGTAAAGTTTGATACGCACATCGTTGACATCGCTGACTGCA
ATAGGAAACTGAAATAGACGGCAAACCATTAGTTCATTCGAAAGAACGTA
TTGTCGAGAATTATCACTCACTATATCAGAAAATTGACACACGAATTATA
TAAACGAAGTTATACAGAAAAAGATTAAAGAAAAGAAA

The DNA sequence of the Fba1 terminator is shown, below, as SEQ ID NO: 20:

GTTAATTCAAATTAATTGATATAGTTTTTTAATGAGTATTGAATCTGTTT
AGAAATAATGGAATATTATTTTTATTTATTTATTTATATTATTGGTCGGC
TCTTTTCTTCTGAAGGTCAATGACAAAATGATATGAAGGAAATAATGATT
TCTTTTAAAATACAACGTAAGATATTTTTACAAAAGCCTAGCTCATCTTT
TGTCATGCACTATTTTACTCACGCTTGAAATTAACGGCCAGTCCACTGCG
GAGTCATTTCAAAGTCATCCTAATCGATCTATCGTTTTTGATAGCTCATT
TTG

### Example 4

### Generation of Yeast Strains with an Additional PTA Expression Cassette

To study the ability of the eight new PTA molecules to affect ethanol, glycerol and acetate production in yeast, the SwaI fragment (see FIG. 1) containing the PTA expression cassette from each plasmid construct described in Example 3 was separately transformed into parental strain FG-PKL. Strain FG-PKL was generated by expression of PKL, PTA, AADH, and ACS in wild-type FERMAX™ Gold strain (Martrex Inc., Minnesota, USA; herein abbreviated, “FG”), a well-known, commercially-available fermentation yeast used in the grain ethanol industry. The FG-PKL strain has been previously described in WO2015148272 (Miasnikov et al.). Transformants were selected and designated FG-PKL followed by a suffix corresponding to the abbreviation for the particular PTA polypeptide listed in Table 1.

### Example 5

### Alcohol Production by Yeast Expressing Different PTA

The FG-PKL strains over-expressing different PTA polypeptides were tested for ethanol, glycerol and acetate production in an Ankom assay. Fermentations were performed at 32° C. for 55 hours. Samples from the end of fermentation were analyzed by HPLC. The results are summarized in Table 3.

TABLE 3
HPLC results from FG-PKL strains

| | Glucose | Glycerol | Acetate | Ethanol | Rel. ethanol |
|---|---|---|---|---|---|
| Strain | (g/L) | (g/L) | (g/L) | (g/L) | increase (%) |
| FG-PKL (parent) | 0.57 | 12.45 | 1.56 | 142.96 | -0- |
| FG-PKL-LpPTA1 | 0.70 | 12.12 | 1.66 | 143.85 | 0.62 |
| FG-PKL-LpPTA2 | 0.74 | 12.21 | 1.63 | 143.83 | 0.61 |
| FG-PKL-LaPTA | 0.77 | 12.17 | 1.75 | 143.87 | 0.64 |
| FG-PKL-LbPTA | 0.80 | 12.17 | 1.75 | 143.22 | 0.24 |
| FG-PKL-LfPTA1 | 0.77 | 12.18 | 1.74 | 143.97 | 0.71 |
| FG-PKL-LfPTA2 | 0.59 | 12.08 | 1.65 | 142.89 | −0.05 |
| FG-PKL-LhPTA | 0.76 | 12.15 | 1.65 | 143.54 | 0.41 |
| FG-PKL-LsPTA | 0.84 | 12.48 | 1.61 | 143.43 | 0.33 |
| FG-PKL-LxPTA | 0.84 | 12.22 | 1.71 | 144.02 | 0.74 |

Expression of an extra copy of almost all PTA polypeptides (except LfPTA2) increased ethanol production compared to the FG-PKL control. Expression of LpPTA1 (SEQ ID NO: 1) and LpPTA2 (SEQ ID NO: 2) resulted in a similar 0.61-0.62% increase in ethanol production. However, expression of LaPTA (SEQ ID NO: 3), LfPTA1 (SEQ ID NO: 5) and LxPTA (SEQ ID NO: 9) resulted in increased ethanol production compared to expression of additional LpPTA1 in engineered cells with PKL pathway.

### Example 6

### Biochemical Characterization of Yeast Strains with Extra Copy of a PTA Expression Cassette

To further characterize FG-PKL strains with an extra PTA expression cassette derived from different organisms, PTA activity was measured directly using the assay described in Example 1. The data are summarized in Table 4.

TABLE 4
PTA enzymatic activity in FG-PTA strains

| | | Increase in PTA | Increased PTA |
|---|---|---|---|
| | PTA activity | activity over | activity over |
| Strain | (μmol/mg/min) | parent (%) | LpPTA1 (%) |
| FG-PKL (parent) | 1.05 | -0- | −71.5 |
| FG-PKL-LpPTA1 | 2.73 | 260 | -0- |
| FG-PKL-LpPTA2 | 2.10 | 238 | −33.1 |
| FG-PKL-LaPTA | 3.45 | 329 | 26 |
| FG-PKL-LbPTA | 3.59 | 342 | 32 |
| FG-PKL-LfPTA1 | 6.21 | 591 | 127 |
| FG-PKL-LfPTA2 | 2.31 | 220 | −15.4 |
| FG-PKL-LhPTA | 2.39 | 228 | −12.5 |
| FG-PKL-LsPTA | 1.16 | 10 | −58 |
| FG-PKL-LxPTA | 3.73 | 355 | 37 |

The results show that expression of LaPTA (SEQ ID NO: 3), LfPTA1 (SEQ ID NO: 5) and LxPTA (SEQ ID NO: 9) resulted in the highest level of PTA activity, consistent with the results described in Example 5. However, in the direct PTA assay, LbPTA (SEQ ID NO: 4) also shows a high level of PTA activity. The reason that LbPTA did not produce increased ethanol levels in Example 5 is unclear.

What is claimed is:

1. Modified yeast cells comprising an exogenous phophoketolase pathway including a phosphotransacetylase gene derived from Lactobacillus acidifarinae, Lactobacillus fabifermentans and/or Lactobacillus xiangfangensis, and/or encoding a polypeptide having at least at least 75% amino acid sequence identity to SEQ ID NO: 3, at least 77% amino acid sequence identity to SEQ ID NO: 5 and/or at least 96% amino acid sequence identity to SEQ ID NO: 9, wherein the phosphotransacetylase gene does not encode a polypeptide identical to the phosphotransacetylase polypeptide from Lactobacillus plantarum having the amino acid sequence of SEQ ID NO: 1.

2. The modified cells of claim 1, wherein the exogenous phophoketolase pathway includes a gene encoding a phosphoketolase and a gene encoding a phosphotransacetylase.

3. The modified cells of claim 2, wherein the exogenous phophoketolase pathway further includes a gene encoding an acetylating acetyl dehydrogenase.

4. The modified cells of any of claims 1-3, further comprising an exogenous gene encoding a carbohydrate processing enzyme.

5. The modified cells of any of claims 1-4, further comprising an alteration in the glycerol pathway and/or the acetyl-CoA pathway.

6. The modified cells of any of claims 1-5, further comprising an alternative pathway for making ethanol.

7. The modified cells of any of claims 1-6, wherein the cells are of a Saccharomyces spp.

8. A method for increasing the production of ethanol from yeast cells grown on a carbohydrate substrate, comprising: introducing into parental yeast cells an exogenous phophoketolase pathway comprising a phosphotransacetylase gene derived from Lactobacillus acidifarinae, Lactobacillus fabifermentans and/or Lactobacillus xiangfangensis, and/or encoding a polypeptide having at least at least 75% amino acid sequence identity to SEQ ID NO: 3, at least 77% amino acid sequence identity to SEQ ID NO: 5 and/or at least 96% amino acid sequence identity to SEQ ID NO: 9, wherein the phosphotransacetylase gene does not encode a polypeptide identical to the phosphotransacetylase polypeptide from Lactobacillus plantarum having the amino acid sequence of SEQ ID NO: 1.

9. The method of claim 8, wherein the phosphotransacetylase gene is in addition to a phosphotransacetylase gene encoding a phosphotransacetylase polypeptide from Lactobacillus plantarum having the amino acid sequence of SEQ ID NO: 1.

10. The method of claim 8 or 9, wherein the yeast cells are the modified yeast cells of any one of claim 1-7.
